(19) United States
(12) Patent Application Publication
(10) Pub. No.: US 2015/0265630 A1
(43) Pub. Date: Sep. 24, 2015

(54) 6-Substituted Estradiol Derivatives for the Treatment of Alzheimer's Disease

(71) Applicant: Endece LLC, Mequon, WI (US)

(72) Inventors: James G. Yarger, Cedarburg, WI (US); Steven H. Nye, Mequon, WI (US)

(21) Appl. No.: 14/663,043

(22) Filed: Mar. 19, 2015

Related U.S. Application Data
(60) Provisional application No. 61/955,578, filed on Mar. 19, 2014.

(51) Int. Cl. A61K 31/565

(52) U.S. Cl. CPC A61K 31/565

(57) ABSTRACT

Disclosed is a method of treating Alzheimer's Disease with 6-substituted estradiol compounds of the formula

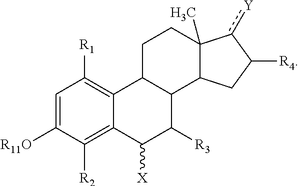

The methods can be used to treat a variety of diseases related to the up-regulation of lipoprotein lipase or apolipoprotein C2.

This application claims priority to and the benefit of provisional patent application Ser. No. 61/955,578 filed Mar. 19, 2014, the entirety of which is incorporated herein by reference.

### FIELD OF THE INVENTION

The present invention relates to a method of treating Alzheimer's Disease (AD) with 6-substituted estradiol compounds and their pharmaceutically acceptable salts or prodrugs. The compounds provide for up-regulation of lipoprotein lipase (LPL) and/or apoliprotein C2 (ApoC2), the protein that activates LPL.

### BACKGROUND OF THE INVENTION

Alzheimer's Disease (AD) is a progressive neurodegenerative disorder which primarily affects the elderly. There are two forms of AD, early-onset and late-onset. Early-onset AD is rare, strikes susceptible individuals as early as the third decade, and is frequently associated with mutations in a small set of genes. Late onset, or spontaneous AD is common, strikes in the seventh or eighth decade, and is a multifactorial disease with many genetic risk factors. Late-onset AD is the leading cause of dementia in persons over the age of 65. An estimated 7-10% of the American population over 65, and up to 40% of the American population greater than 80 years of age is afflicted with AD. Early in the disease, patients experience loss of memory and orientation. As the disease progresses, additional cognitive functions become impaired, until the patient is completely incapacitated.

It has been widely accepted that the accumulation of amyloid-beta (Aβ) peptides in the brain extracellular space and alteration in Aβ molecular composition are critical for developing synaptic and cognitive deficits in AD. Aβ is produced by sequential limited proteolysis of the amyloid precursor protein (APP) by two aspartyl proteases, beta and gamma-secretases. Proteolysis by gamma-secretase is the last processing step resulting in release of Aβ. Normally, gamma-secretase cleavage results in Aβ proteins of 40 amino acids in length (Aβ40) and 42 amino acids in length (Aβ42), the latter being the predominant species in senile plaques (Iwatsubo, T., et al, 1994 Neuron 13, 45-53). The Aβ levels are determined by the balance between its production and degradation/clearance, and an attenuated Aβ catabolism is suggested to cause Aβ accumulation in aging brains (Tanzi, R. E., et al, 2004 Neuron 43, 605-608). Previous studies have shown that astrocytes and microglia directly take up and degrade Aβ42 (Wyss-Coray, T., et al, 2003 Nat. Med. 9, 453-457; Jiang, Q., et al. 2008 Neuron 58, 681-693) and that Aβ degradation occurs in endosomal-lysosomal compartments (Majumdar, A., et al, 2007 Mol. Biol. Cell 18, 1490-1496; Mandrekar, S., 2009 J. Neurosci. 29, 4252-4262).

Lipoprotein lipase (LPL) catalyzes the hydrolysis of triglycerides and mediates the cellular uptake of lipoproteins by functioning as a “bridging molecule” between lipoproteins and sulfated glycosaminoglycans (GAGs) or lipoprotein receptors in blood vessels (Williams, K. J., et al, 1992 J. Biol. Chem. 267, 13284-13292; Mulder, M., et al, 1993 J. Biol. Chem. 268, 9369-9375). Sulfated GAGs are side chains of proteoglycans normally found in the extracellular matrix and on the cell surface in the peripheral tissues, e.g. adipose, heart and skeletal muscle tissue, and brain. The role of LPL in the brain is, to date, unknown.

Interestingly, it has been shown that LPL is accumulated in senile plaques of AD brains (Rebeck, G. W., et al, 1995 Ann. Neurol. 37, 211-217). Moreover, single nucleotide polymorphisms (SNPs) in the coding region of the LPL gene are associated with disease incidence in clinically diagnosed AD subjects, LPL mRNA expression level, brain cholesterol level, and the severity of AD pathologies, including neurofibrillary tangles and senile plaque density (Blain, J. F., et al, 2006 Eur. J. Neurosci. 24, 1245-1251). These results suggest that LPL may have a physiological role in the brain, whose alternation is associated with the pathogenesis of AD.

Recently, researchers carried out experiments to determine whether LPL interacts with Aβ to promote Aβ cellular uptake and degradation in astrocytes, and found evidence that LPL forms a complex with Aβ and facilitates Aβ cell surface binding and uptake in mouse primary astrocytes through a mechanism that is dependent on heparan sulfate and chondroitin sulfate GAG chains, leading to the lysosomal degradation of Aβ (Nishitsuji, K., et al, 2011 J. Biol. Chem. 286(8), 6393-6401). In addition, it was found that enhanced cyclin-dependent kinase 5 (CDK5) activity contributed to LPL up-regulation and promoted Aβ phagocytosis in microglia, whereas inhibition of CDK5 reduced LPL expression and Aβ internalization. Thus, a viable treatment for AD is the up-regulation of LPL (and/or ApoC2, or apoliprotein C2, the protein that activates LPL). To this end, compounds such as statins have been shown to stimulate LPL activity and therefore perhaps could play a role in the pathogenesis of AD (Schoonjans, K., et al, 1999 FEBS Lett 452, 160-164; Mead, J., et al, 2002 J. Mol. Med. 80, 753-769).

This would be a significant improvement over current treatment of AD. The only drugs to treat AD on the market today, Aricept®, Cognex®, Reminyl® and Exelon® are acetylcholinesterase inhibitors. These drugs do not address the underlying pathology of AD. They merely enhance the effectiveness of those nerve cells still able to function and only provide symptomatic relief from the disease. Since the disease continues, the benefits of these treatments are slight.

Accordingly, disclosed herein are certain 6-substituted estradiol derivatives that provide for the up-regulation of LPL (and/or ApoC2). This is found surprising in that estrogen itself has previously been reported to markedly decrease the amounts of fat accumulation and LPL mRNA as well as triglyceride accumulation in genetically manipulated 3T3-L1 adipocytes stably expressing the estrogen receptor (ER) (Homma, H., et al, 2000 J. Biol. Chem. 275(15), 11404-11411. As presented below, the instant disclosures proved a different and surprising approach for the treatment of AD, thereby addressing the deficiencies presented in the prior art.

### FIELD OF THE INVENTION

In light of the foregoing, it is an object of the present invention to provide a method of treating a patient with Alzheimer's Disease comprising administering to a patient in need thereof a therapeutically effective amount of a 6-substituted estradiol derivative. The 6-substituted estradiol derivatives of the invention provide for the up-regulation of lipoprotein lipase (LPL) and/or apoliprotein C2 (ApoC2). It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

Accordingly, in one aspect of the invention, the 6-substituted estradiol derivatives used in the methods disclosed herein are a compound of the formula I:

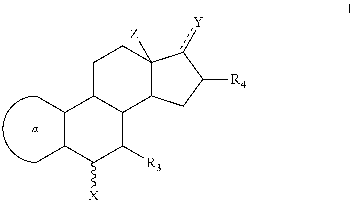

wherein the “a” ring is selected from the group consisting of

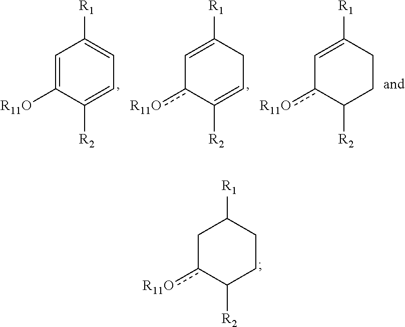

R₁, R₂, R₃ and R₄ are independently hydrogen, C₁-C₆ alkyl, halo, a sulfate, a glucuronide, —OH, a bulky group, aryl, cycloalkyl, heteroaryl, heterocycloalkyl, —N(CH₂)ₙ; a phosphate group, and a phosphinate group; R₁₁ is selected from the group consisting of H, C₁-C₆ alkyl, halogen, a sulfate, a glucoronide, —SO₂NH₂, —COOH, —CN, —CH₂CN—, —NHCN—, —CHO, ═CHOCH₃, —COO salt, —OSO₂alkyl, —NH₂, and —NHCO(CH₂)ₙ; X is selected from the group consisting of C₁-C₁₂ alkyl, C₂-C₁₂ alkenyl, C₂-C₁₂ alkynyl, halogen, a glucoronide, —NH₂, —SO₂NH₂, —COOH, —CN, —CH₂CN, —NHCN, —CHO, —COOsalt, —OSO₂alkyl, —SH, —SCH₃, —CH[(CH₂)ₙCH₃]COOCH₃, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—NH—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)CH₃, —(CH₂)ₘ—OH, —(CH₂)ₘ—NH₂, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —N(CH₃)₂, —(CH₂)ₘ(NH)CH₂OH, —NHCOOH, —(CH₂)ₘNHCOOH, —NO₂, —SCN, —SO₂alkyl, —B(OH)₂, —(CH₂)ₘ N(CH₃)—SO₂—NH₃, —(CH₂)ₘ—NH—SO₂—NH₂, —NHC(═S)CH₃, and —NHNH₂; Y is selected from hydrogen, ═O, —OCO(C₁-C₂₀ alkyl) and —OH; and Z is H or methyl; wherein m is an integer between 0-20, n is an integer between 0-8, the [symbol] symbol represents either a single or a double bond capable of forming a keto group at position 3 and/or 17; and the [symbol] symbol represents any type of bond regardless of the stereochemistry; and the respective enantiomers, other stereochemical isomers, hydrates, solvates, tautomers and pharmaceutically acceptable salts of said compounds.

In another aspect of the invention, the method specifically provides for compounds that bind to one or both of estrogen receptor-α (ER-α) and estrogen receptor-β (ER-β). Such a method can comprise initiating, enhancing or increasing gene transcription for RNA encoding genes involved in key signaling pathways for the expression of LPL and/or ApoC2.

Other objects, features, benefits and advantages of the present invention will be apparent from this summary and the following descriptions of certain embodiments, and will be readily apparent to those skilled in the art having knowledge of various steroid compounds and related therapeutic methods. Such objects, features, benefits and advantages will be apparent from the above as taken into conjunction with the accompanying examples, data, figures or with consideration of the references incorporated herein.

### DETAILED DESCRIPTION OF THE INVENTION

Unless defined otherwise, all technical and scientific terms used herein have the same meaning as is commonly understood by one of skill in the art to which this invention belongs and shall be understood to have the meanings described below. All publications and patents referred to herein are incorporated by reference in their entirety. Unless otherwise specified, a reference to a particular compound includes all such isomeric forms, including racemic and other mixtures thereof. Unless otherwise specified, a reference to a particular compound also includes ionic, salt, solvate (e.g., hydrate), protected forms, prodrugs, and other stereoisomers thereof, for example, as discussed herein.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, and discussed herein.

The term “treatment,” or “therapy” as used herein in the context of treating a condition, pertains generally to treatment and therapy of a mammalian subject, whether of a human or a non-human animal (e.g., in veterinary applications), in which some desired therapeutic effect is achieved, for example, the inhibition of the progress of the condition, and includes a reduction in the rate of progress, a halt in the rate of progress, amelioration of the condition, and/or cure of the condition. Treatment as a prophylactic measure is also included. Treatment includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), anti-inflammatory, prodrugs (e.g., employing protecting groups including phosphoric acid derivatives and phosphinates at suitable positions such as position 3 or 17, other compounds used for photodynamic therapy, GDEPT, ADEPT, etc.), surgery, radiation therapy, and gene therapy.

The term “stereochemical isomer” as used herein, refers to isomers that differ from each other only in the way the atoms are oriented in space. The two stereoisomers particularly of importance in the instant invention are enantiomers and diastereomers depending on whether or not the two isomers are mirror images of each other. In the preferred embodiment, the claimed formulations comprise such compounds that isolated, resolved and are “substantially free of other isomers.”

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio. By the term “effective amount” is meant an amount that can bring about a detectable effect, generally.

The term “patient” or “subject” refers to animals, including mammals, preferably humans.

The term “tissue” refers generally to specialized cells which may perform a particular function. The term “tissue” may refer to an individual cell or a plurality or aggregate of cells, for example, membranes, blood or organs. The term “tissue” also includes reference to an abnormal cell or a plurality of abnormal cells. Exemplary tissues include breast tissue, including breast cells, membranous tissues, including endothelium and epithelium, laminae, connective tissue, including interstitial tissue, brain tissue and tumors.

By “alkyl” in the present invention is meant a straight or branched chain alkyl radical having 1-20, and preferably from 1-12, carbon atoms. Examples include but are not limited to methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. Each alkyl group may be optionally substituted with one, two or three substituents such as, for example, a halo, cycloalkyl, aryl, alkenyl or alkoxy group and the like.

By “aryl” is meant an aromatic carbocylic radical having a single ring (e.g. phenyl), multiple rings (e.g. biphenyl) or multiple fused rings in which at least one is aromatic (e.g. 1,2,3,4-tetrahydronaphthyl). The aryl group can also be optionally mono-, di-, or trisubstituted with, for example, halo, alkyl, alkenyl, cycloalkyl or alkoxy and the like.

By “heteroaryl” is meant one or multiple fused aromatic ring systems of 5-, 6- or 7-membered rings containing at least one and up to four heteroatoms selected from nitrogen, oxygen or sulfur. Examples include but are not limited to furanyl, thienyl, pyridinyl, pyrimidinyl, benzimidazolyl and benzoxazolyl. The heteroaryl group can also be optionally mono-, di-, or trisubstituted with, for example, halo, alkyl, alkenyl, cycloalkyl or alkoxy and the like.

By “cycloalkyl” is meant a carbocylic radical having a single ring (e.g. cyclohexyl), multiple rings (e.g. bicyclohexyl) or multiple fused rings (e.g.). The cycloalkyl group can optionally contain from 1 to 4 heteroatoms. In addition, the cycloalkyl group may have one or more double bonds. The cycloalkyl group can also be optionally mono-, di-, or trisubstituted with, for example, halo, alkyl, alkenyl, aryl or alkoxy and the like.

By “alkoxy” is meant an oxy-containing radical having an alkyl portion. Examples include, but are not limited to, methoxy, ethoxy, propoxy, butoxy and tert-butoxy. The alkoxy group can also be optionally mono-, di-, or trisubstituted with, for example, halo, aryl, cycloalkyl or alkoxy and the like.

By “alkenyl” is meant a straight or branched hydrocarbon radical having from 2 to 20, and preferably from 2-6, carbon atoms and from one to three double bonds and includes, for example, ethenyl, propenyl, 1-but-3-enyl, 1-pent-3-enyl, 1-hex-5-enyl. The alkenyl group can also be optionally mono-, di-, or trisubstituted with, for example, halo, aryl, cycloalkyl or alkoxy and the like.

“Halo” or “halogen” is a halogen radical of fluorine, chlorine, bromine or iodine.

By “glucuronide” is meant a glycoside radical of glucuronic acid.

The term “sulfate” refers to a radical having the general formula —OS(O)₂—OR′, wherein R′ is hydrogen, a metal or an alkyl group.

The term “phosphate” refers to a radical having the general formula —OP(O)(OR′)₂, wherein each R′ is independently hydrogen, a metal or an alkyl group.

The term “phosphinate” refers to a radical having the general formula —OP(O)(R′)₂, wherein each R′ is independently hydrogen, a metal or an alkyl group.

By “bulky group” is meant a substituent that produces steric hindrance about the space to which it is attached, e.g. a t-butyl group.

The term “amino alkyl” as used herein refers to an alkyl group with an amino group on it, for example, H₂N—CH₂—, H₂N—CH₂CH₂—, Me₂NCH₂—, etc., wherein the point of attachment is a carbon of the alkyl chain; and the term “alkyl amino” as used herein refers to an amino group with an alkyl group attached to the nitrogen atom, for example, CH3NH—, EtNH—, iPr—NH—, etc., wherein the point of attachment is via the nitrogen atom of the amino group. All other terms wherein successive radicals are employed will adhere to a similar rule.

In an embodiment of the invention, a method of treating or preventing Alzheimer's Disease in a patient comprising administering to a patient in need thereof a therapeutically effective amount of a 6-substituted estradiol derivative of Formula I is described. Preferably, the method provides up-regulation of LPL and/or ApoC2 functional activity in a mammal, said method comprising administering to said mammal an effective amount of a compound of Formula I.

Other disorders that involve the build-up of amyloid plaques can also be treated by the compounds of Formula I, and include but are not limited to, for example, Lewy body dementia, inclusion body myositis, and cerebral amyloid angiopathy.

In an embodiment of the present invention, compounds of the methods have the general structure shown in Formula (Ia) below:

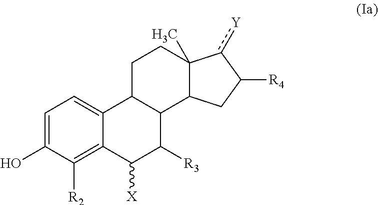

wherein R₂, R₃, R₄, X and Y are as defined above for Formula (I). Even more preferably, Y is selected from ═O and —OH; R₄ is selected from hydrogen, halo and C₁-C₆ alkyl; R₂ is selected from hydrogen, —OH and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; m is an integer from 1-20; n is an integer from 0-8; and the [symbol] symbol represents either a single or a double bond. Yet even more preferably, Y is (S)—OH; R₄ is selected from hydrogen or alkyl; R₂ is hydrogen; R₃ is hydrogen; and X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-12; n is an integer from 0-4; and the C-13 methyl is in the (S) configuration.

Yet another embodiment of the present invention is directed to methods using compounds of a Formula (Ib):

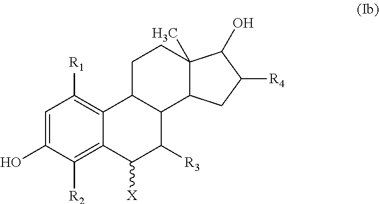

wherein R₁R₂, R₃, R₄ and X are as defined above for Formula (I). Even more preferably, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘN—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘ N(CH₃)—SO₂—NH₃, and —(CH₂)ₘ—NH—SO₂—NH₂; m is an integer from 1-20; and n is an integer from 0-8. Yet even more preferably, R₁ is hydrogen; R₄ is selected from hydrogen or alkyl; R₂ is hydrogen; R₃ is hydrogen; and X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-12; n is an integer from 0-4; and both the C-13 methyl and C-17 hydroxyl are in the (S) configuration.

Still another embodiment of the invention is directed to methods using a compound of a Formula (Ic):

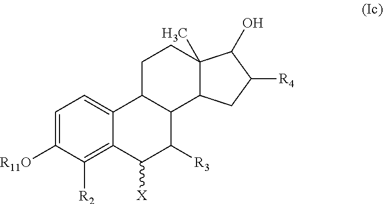

wherein R₁₁, R₂, R₃, R₄ and X are as defined above for Formula (I). Even more preferably, R₁₁ is hydrogen or C₁-C₆ alkyl; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘN—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘ N(CH₃)—SO₂—NH₃, and —(CH₂)ₘ—NH—SO₂—NH₂; m is an integer from 1-20; and n is an integer from 0-8. Yet even more preferably, R₁₁ is hydrogen; R₄ is selected from hydrogen or alkyl; R₂ is hydrogen; R₃ is hydrogen; and X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-12; n is an integer from 0-4; and both the C-13 methyl and C-17 hydroxyl are in the (S) configuration.

Yet another embodiment of the present invention is directed to methods using a compound of a Formula (Id):

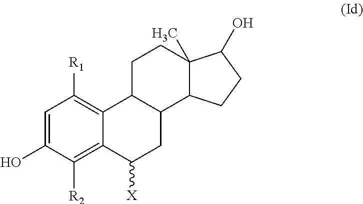

wherein R₁, R₂, and X are as defined above for Formula (I). Even more preferably, R₁ is selected from hydrogen, —OH and halo; R₂ is selected from hydrogen and halo; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘ N(CH₃)—SO₂—NH₃, and —(CH₂)ₘ—NH—SO₂—NH₂; X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-20; and n is an integer from 0-8. Still even more preferably, R₁ and R₂ are hydrogen; m is an integer from 1-12; n is an integer from 0-4; and both the C-13 methyl and C-17 hydroxyl are in the (S) configuration.

Yet another embodiment of the present invention is directed to methods using a compound of a Formula (Ie):

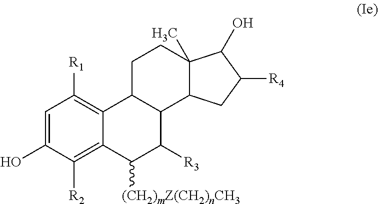

wherein m, n, R₁, R₂, R₃ and R₄ are as defined above for Formula (I), and Z is selected from —O—, —S— and —NH—. Even more preferably, m is 1-12, n is 0-4, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; Z is selected from —O— and —S—; and both the C-13 methyl and C-17 hydroxyl are in the (S) configuration.

Still another embodiment of the present invention is directed to methods using a compound of a Formula (If):

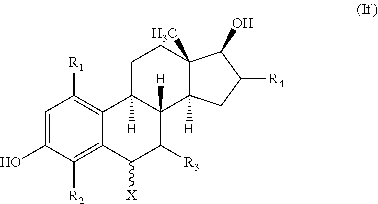

wherein R₁, R₂, R₃, R₄ and X are as defined above for Formula (I). Even more preferably, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘ N(CH₃)—SO₂—NH₃, and —(CH₂)ₘ—NH—SO₂—NH₂; X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-20; and n is an integer from 0-8. Still even more preferably, R₁, R₂, R₃ and R₄ are hydrogen; m is an integer from 1-12; and n is an integer from 0-4.

Still another embodiment of the present invention is directed to methods using a compound of a Formula (Ig):

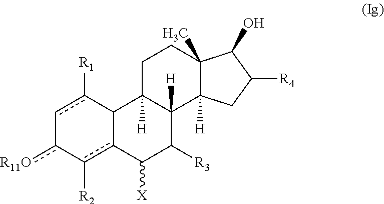

wherein R₁, R₂, R₃, R₄, R₁₁ and X are as defined above for Formula (I). Even more preferably, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘ N(CH₃)—SO₂—NH₃, and —(CH₂)ₘ—NH—SO₂—NH₂; X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-20; [symbol] OR₁₁ is either ═O or —OH; and n is an integer from 0-8. Still even more preferably, R₁, R₂, R₃ and R₄ are hydrogen; m is an integer from 1-12; and n is an integer from 0-4.

Specific examples of compounds of Formula (I) and (Ia)-(If) are shown below:

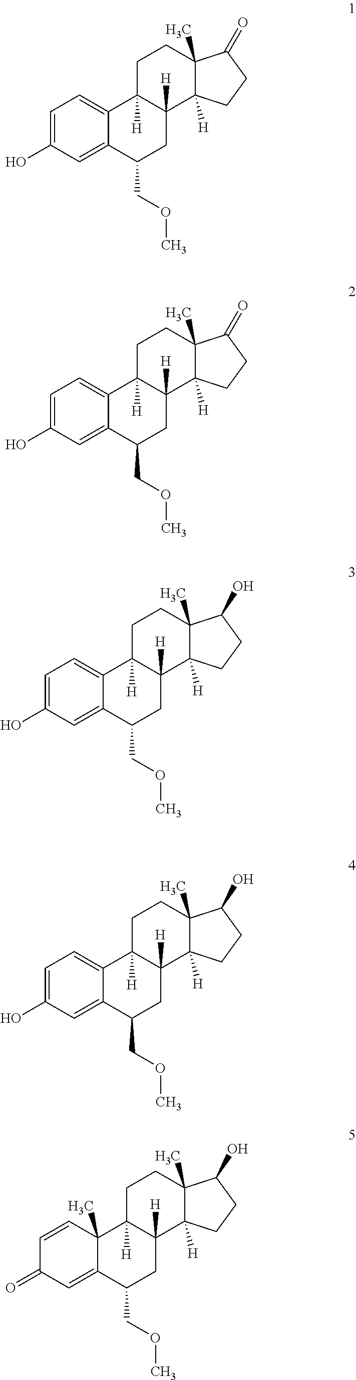

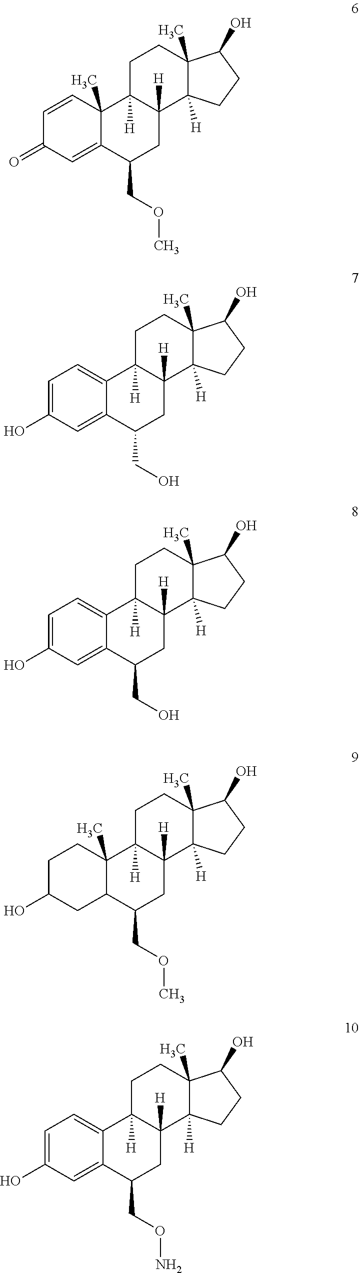

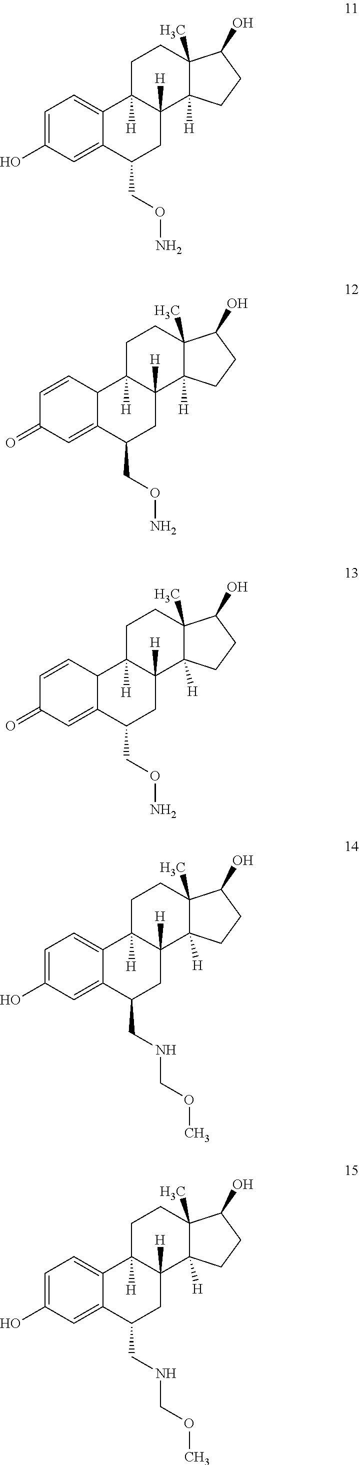

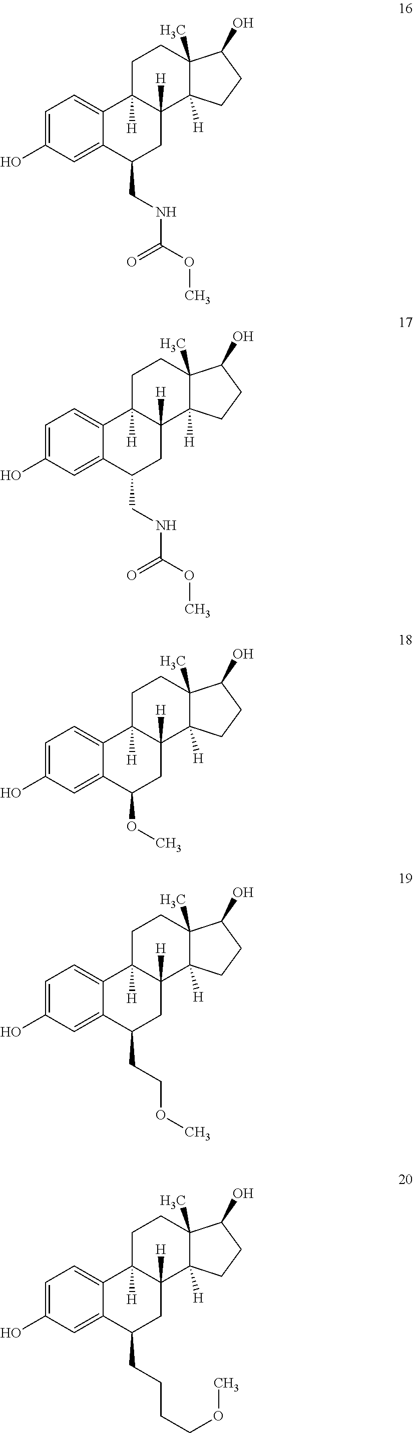

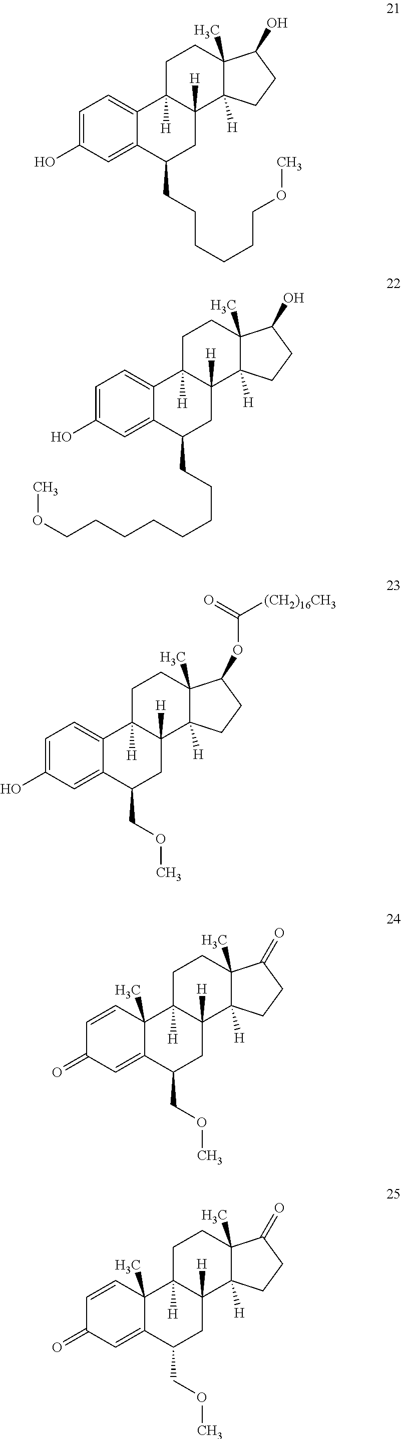

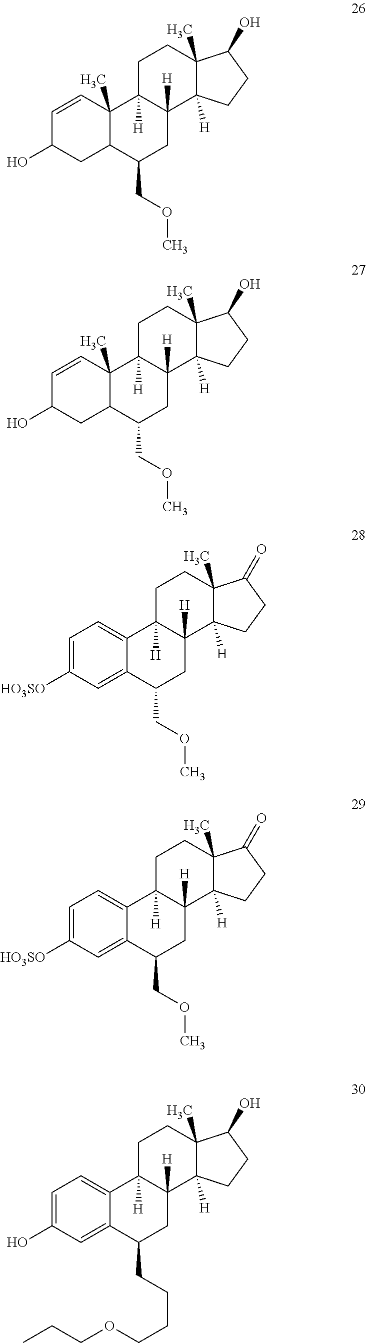

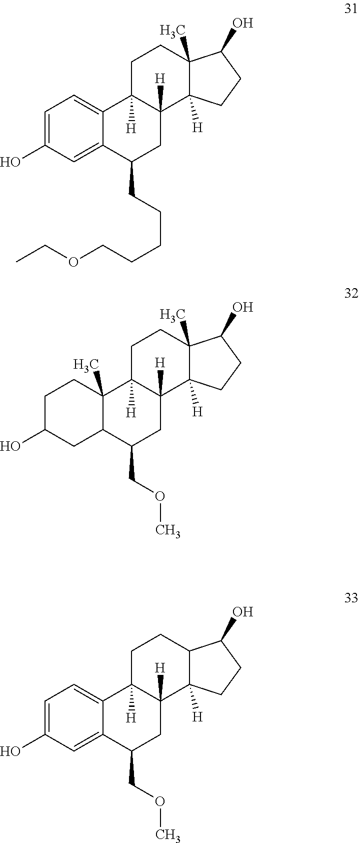

Embodiment compounds of the present invention can be used in a pharmaceutical composition. Such a composition can comprise one or more compounds selected from those discussed above, illustrated below or otherwise inferred herein, and combinations thereof. In certain embodiments, such a composition can comprise a pharmaceutically-acceptable carrier component. Without limitation, such a composition can comprise a racemic mixture of compounds. In certain embodiments, such a compound can be present as the S and R enantiomer, preferably its isolated and purified form which is substantially free of the other isomer.

The compounds of the present invention may have asymmetric centers and may occur as a racemate, a racemic mixture or as individual and purified diastereomers or enantiomers such as (named via ChemDraw Ultra, Version 11.0(3) or 12.0) (6S,8R,9S,13S,14S)-3-hydroxy-6-(methoxymethyl)-13-methyl-7,8,9,11,12,13,15,16-octahydro-6H-cyclopenta[a]phenanthren-17(14H)-one (compound 1); (6R,8R,9S,13S,14S)-3-hydroxy-6-(methoxymethyl)-13-methyl-7,8,9,11,12,13,15,16-octahydro-6H-cyclopenta[a]phenanthren-17(14H)-one (compound 2); (6S,8R,9S,13S,14S)-6-(methoxymethyl)-13-methyl-7,8,9,11,12,13,14,15,16,17-decahydro-6H-cyclopenta[a]phenanthrene-3,17-diol (compound 3); (6R,8R,9S,13S,14S)-6-(methoxymethyl)-13-methyl-7,8,9,11,12,13,14,15,16,17-decahydro-6H-cyclopenta[a]phenanthrene-3,17-diol (compound 4); (6S,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one (compound 5); (6R,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one (compound 6); (6S,8R,9S,13S,14S)-6-(hydroxymethyl)-13-methyl-7,8,9,11,12,13,14,15,16,17-decahydro-6H-cyclopenta[a]phenanthrene-3,17-diol (compound 7); (6R,8R,9S,13S,14S)-6-(hydroxymethyl)-13-methyl-7,8,9,11,12,13,14,15,16,17-decahydro-6H-cyclopenta[a]phenanthrene-3,17-diol (compound 8); (6R,8R,9S,10R,13S,14S)-6-(methoxymethyl)-10,13-dimethylhexadecahydro-1H-cyclopenta[a]phenanthrene-3,17-diol (compound 9); (6R,8R,9S,13S,14S)-6-((aminooxy)methyl)-13-methyl-7,8,9,11,12,13,14,15,16,17-decahydro-6H-cyclopenta[a]phenanthrene-3,17-diol (compound 10); (6S,8R,9S,13S,14S)-6-((aminooxy)methyl)-13-methyl-7,8,9,11,12,13,14,15,16,17-decahydro-6H-cyclopenta[a]phenanthrene-3,17-diol (compound 11); (6R,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one (compound 12); (6S,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one (compound 13); (6R,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol (compound 14); (6S,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol (compound 15); 1-((((6R,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one (compound 16); 1-((((6S,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one (compound 17); (6R,8R,9S,13S,14S)-6-methoxy-13-methyl-7,8,9,11,12,13,14,15,16,17-decahydro-6H-cyclopenta[a]phenanthrene-3,17-diol (compound 18); (6S,8R,9S,13S,14S)-6-(2-methoxyethyl)-13-methyl-7,8,9,11,12,13,14,15,16,17-decahydro-6H-cyclopenta[a]phenanthrene-3,17-diol (compound 19); (6R,8R,9S,13S,145)-6-(4-methoxybutyl)-13-methyl-7,8,9,11,12,13,14,15,16,17-decahydro-6H-cyclopenta[a]phenanthrene-3,17-diol (compound 20); (6R,8R,9S,13S,14S)-6-(6-methoxyhexyl)-13-methyl-7,8,9,11,12,13,14,15,16,17-decahydro-6H-cyclopenta[a]phenanthrene-3,17-diol (compound 21); (6R,8R,9S,13S,14S)-6-(6-methoxyoctyl)-13-methyl-7,8,9,11,12,13,14,15,16,17-decahydro-6H-cyclopenta[a]phenanthrene-3,17-diol (compound 22); (6R,8R,9S,13S,14S)-3-hydroxy-6-(methoxymethyl)-13-methyl-7,8,9,11,12,13,14,15,16,17-decahydro-6H-cyclopenta[a]phenanthren-17-yl stearate (compound 23); (6R,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione (compound 24); (6S,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione (compound 25); (6R,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol (compound 26); (6S,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol (compound 27); (6S,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate (compound 28); (6R,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate (compound 29); (6R,8R,9S,13S,14S)-13-methyl-6-(4-propoxybutyl)-7,8,9,11,12,13,14,15,16,17-decahydro-6H-cyclopenta[a]phenanthrene-3,17-diol (compound 30); (6R,8R,9S,13S,14S)-13-methyl-6-(5-ethoxypentyl)-7,8,9,11,12,13,14,15,16,17-decahydro-6H-cyclopenta[a]phenanthrene-3,17-diol (compound 31); (6R,8R,9S,10R,13S,14S)-6-(methoxymethyl)-10,13-dimethylhexadecahydro-1H-cyclopenta[a]phenanthrene-3,17-diol (compound 32); and (6R,8S,9S,14S,17S)-6-(methoxymethyl)-7,8,9,11,12,13,14,15,16,17-decahydro-6H-cyclopenta[a]phenanthrene-3,17-diol (compound 33).

The compounds of the methods of the invention are prepared as described in U.S. Ser. No. 12/627,874 (incorporated herein by reference) and pertain to a method for preparing a 6-hydroxymethyl, 6-alkoxyalkyl, 6-alkylthioalkyl, 6-aminomethoxy, 6-methylaminomethoxy, or 6-methoxyamine derivatives of estradiol. Reaction schemes for preparing estradiol derivatives is given below, Schemes 1-3. Such methods can comprise reaction of a t-butyldimethylsilyl derivative of estradiol with LIDAKOR/THF/formaldehyde to obtain a 6-hydroxylated compound followed by such steps as: (i) hydrolysis to obtain 6-hydroxymethyl derivative of estradiol; and/or (ii) treatment with dimethylsulfate followed by hydrolysis to obtain 6-methyloxymethyl derivative of estradiol. Compound 1 can be obtained by further oxidation of compound 3 at the C-17 hydroxyl position. Compound 33 and other dimethyl compounds can be prepared according to U.S. Ser. No. 13/232,798 (incorporated herein by reference).

In an alternative approach, the compounds of the present invention can also be prepared by a method comprising such steps as: (i) protecting an estrodial compound, (ii) acylating the protected estradiol compound at the benzylic 6-position with LIDAKOR/Butyl-Lithium/Diisopropylamine/potassium tert-amylate, (iii) reducing the position 6 aldehyde with lithium aluminum hydride, (iv) deprotecting the protected regions of the estradiol compound. A reaction scheme for preparing estradiol derivatives is given below in Scheme 2.

The compounds of the present invention can be synthesized by the following methods as depicted in the schemes below.

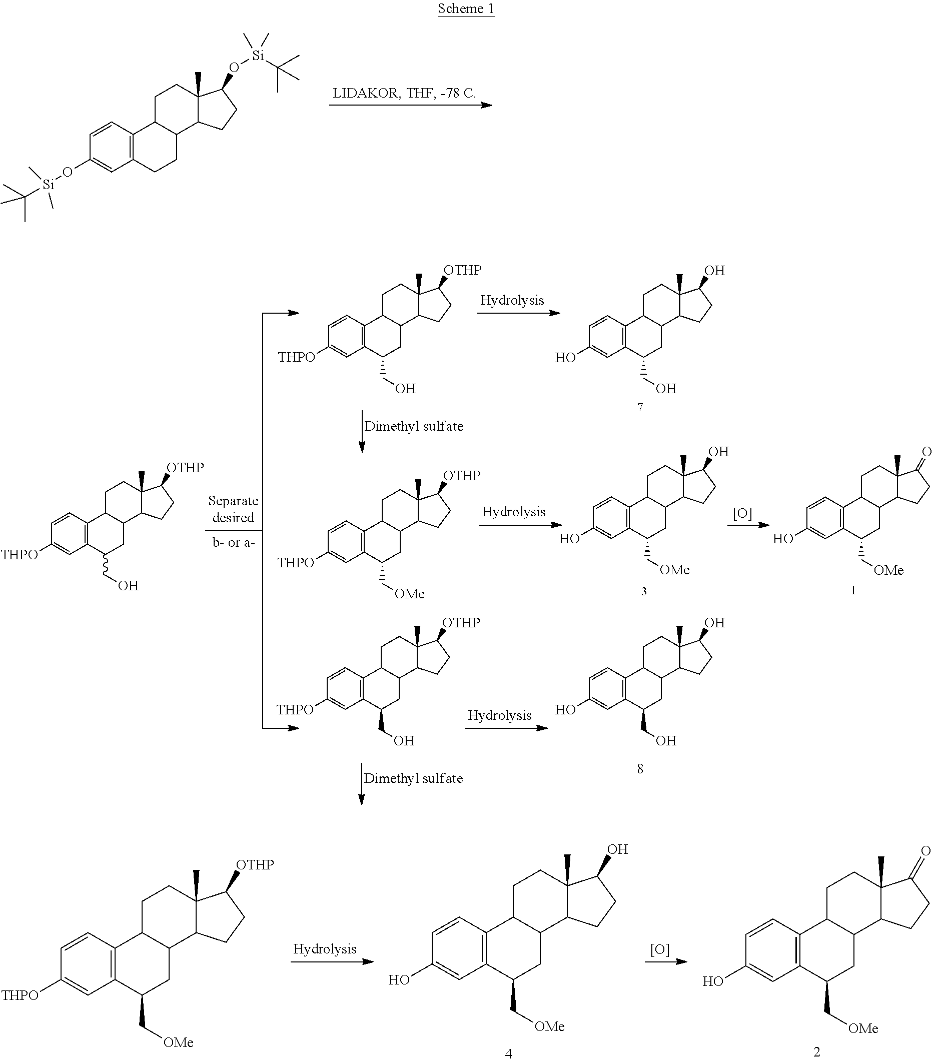

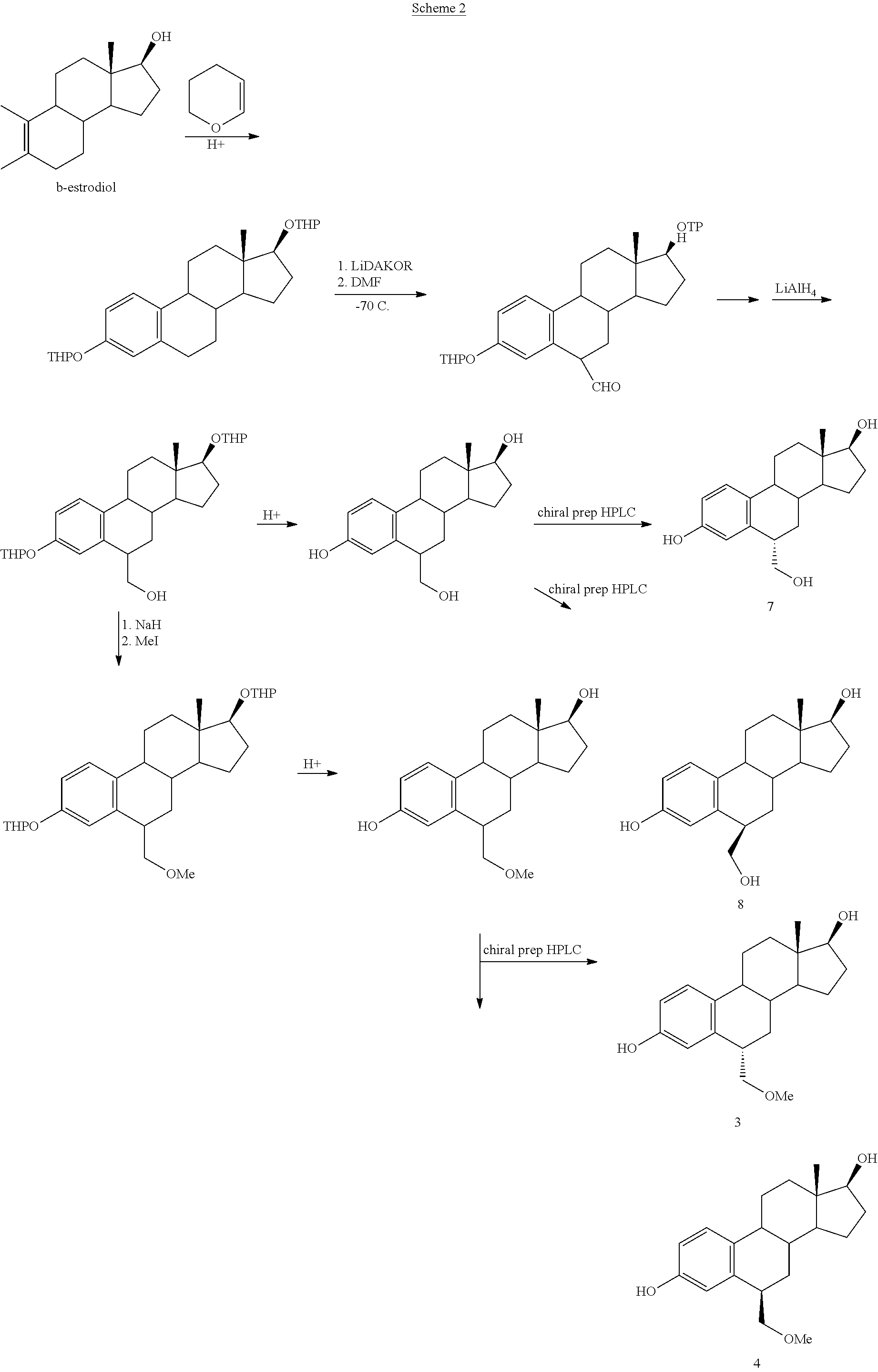

Various alkyloxyalkyl derivatives, in accordance with this invention, involve selection of alkylating agents. Such derivatives would be understood by those skilled in art made aware of this invention, and is available through synthetic procedures of the sort described herein. Accordingly, without limitation, various C₁ to C₆ alkyl and substituted alkyl reagents can be used as described herein to prepare the corresponding alkyloxyalkyl derivatives.

In another aspect of the invention, methods of making 6-amino derivatives of the estradiol are disclosed in reaction schemes below. Accordingly, 6-methoxylated estradiols described in Schemes 1-2 are employed and converted to their respective amino derivatives.

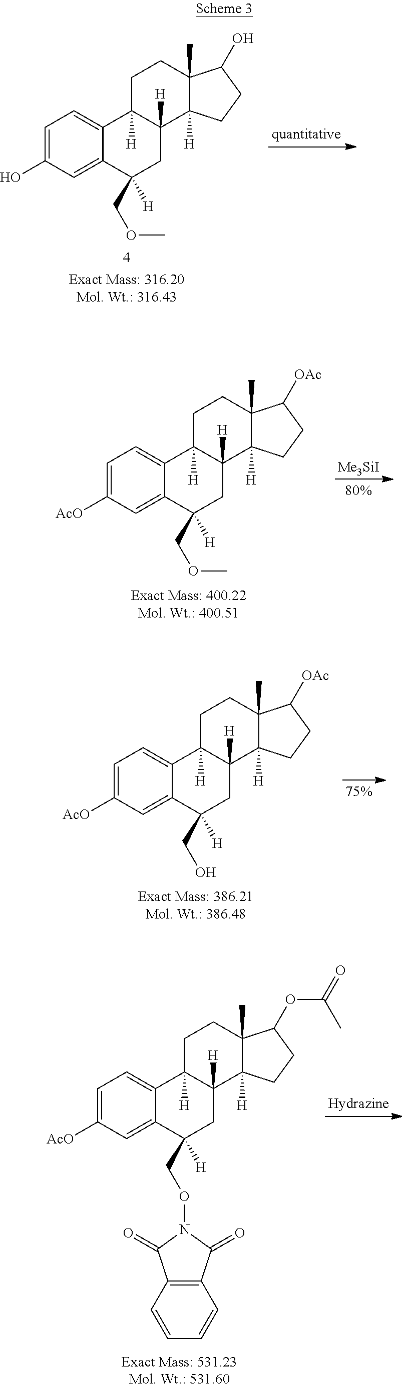

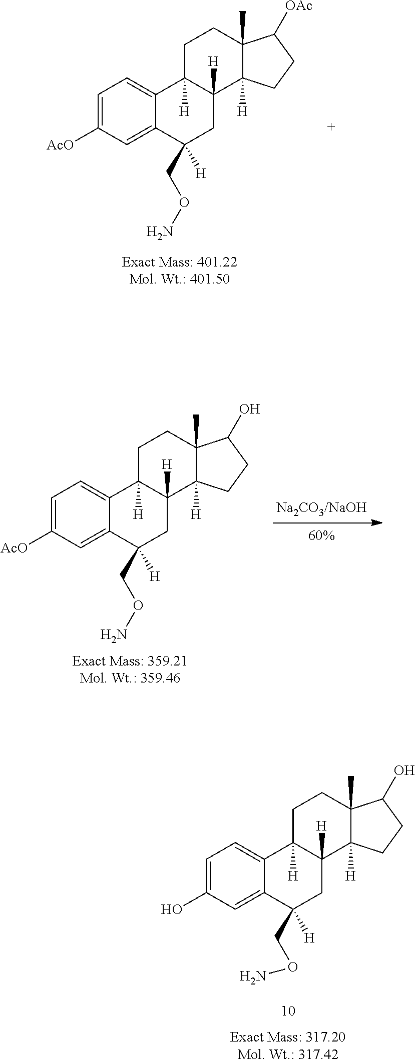

Methods and compounds for preventing Alzheimer's Disease and related disorders are provided. In an aspect of the invention, a method for initiating, enhancing or increasing gene transcription for RNA encoding the LPL protein gene and/or the ApoC2 protein gene in a cell is provided, comprising contacting the cell with an effective amount of a 6-substituted estradiol derivative selected from Formulas (I) and (Ia) to (If). It is to be understood that such initiating, enhancing or increasing of gene transcription can occur for one or more of these genes.

As noted herein, the salts of the compounds of this invention refer to non-toxic “pharmaceutically acceptable salts.” Other salts may, however, be useful in the preparation of the compounds according to the invention or of their pharmaceutically acceptable salts. When the compounds of the present invention contain a basic group, salts encompassed within the term “pharmaceutically acceptable salts” refer to non-toxic salts which are generally prepared by reacting the free base with a suitable organic or inorganic acid. Representative salts include any such salt known in the art. Where compounds of the present invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

As noted herein, the compounds of the present invention can be used in combination with other agents or other agents which will enhance the treatment regime for the mammalian subject. For example, the compounds of the methods could be used in combination with other estrogen receptor-β modulators. The individual components of such combinations can be administered separately at different times during the course of therapy or concurrently in divided or single combination forms to patients or regions of such patients in need of such therapy. The instant invention is therefore to be understood as embracing all such regimes of simultaneous or alternating treatment and the term “administering” is to be interpreted accordingly. It will be understood that the scope of combinations of the compounds of this invention with other agents useful to treat the targeted disease includes in principle any combination with any pharmaceutical composition useful for treating disorders related to estrogen functioning.

The following non-limiting examples and data illustrate various aspects and features relating to the compounds, compositions and/or methods of the present invention, including the synthesis of 6-substituted estradiol derivatives, as are available though the methodologies described herein. In comparison with the prior art, the present compounds and methods provide results and data which are surprising, unexpected and contrary thereto. While the utility of this invention is illustrated through the preparation and use of several compounds, moieties and/or substituents thereof, it will be understood by those skilled in the art that comparable results are obtainable with various other compounds, moieties and/or substituents, as are commensurate with the scope of this invention.

As noted above, the compounds of the methods are prepared according the procedures disclosed in U.S. Ser. No. 12/627,874 and U.S. Ser. No. 13/232,798. To exemplify the synthetic schemes described above and in detail in U.S. Ser. No. 12/627,874, the preparation of compound 21 is provided in Example 1.

### Example 1

### Methods for Preparing Compound 21

a) (8R,9S,13S,14S,17S)-3,17-bis(methoxymethoxy)-13-methyl-7,8,9,11,12,13,14,15,16,17-decahydro-6H-cyclopenta[a]phenanthrene—Chloromethyl methyl ether (7.0 mL, 92.0 mmol) is added to a solution of β-estradiol (5 g, 18.4 mmol) and diisopropylethylamine (16.0 mL 92 mmol) in 100 mL of THF. The reaction mixture is heated to reflux and stirred for 18 hours. The THF is removed in vacuo, and the yellow/brown oil is partitioned between water and CH₂Cl₂. The organic layer is separated, washed with brine, dried (Na₂SO₄), filtered, and evaporated in vacuo to give a golden oil. Purification by silica gel column chromatography (10% EtOAc/Hex) affords the title compound as a viscous, clear oil (5.7 g, 86%).

b) (8R,9S,13S,14S,17S)-3,17-bis(methoxymethoxy)-13-methyl-7,8,9,11,12,13,14,15,16,17-decahydro-6H-cyclopenta[a]phenanthren-6-ol—To a solution of potassium tert-butoxide (8.87 g, 79.0 mmol) and diisopropylamine (11.2 mL, 79.0 mmol) in 80 mL of anhydrous THF cooled to −78° C. under argon is added n-butyllithium (49.4 mL, 79.0 mmol, 1.6 M in hexane) dropwise. The reaction mixture is stirred at −78° C. for 30-45 minutes. A solution of the compound from a) (5.7 g, 15.8 mmol) in 45 mL of THF is then added dropwise, and the reaction mixture is stirred for 3 hours at −78° C. During the addition of the compound from a), the reaction turns a deep red color. Trimethyl borate (10.6 mL, 94.8 mmol) is then added slowly, and the mixture is warmed to 0° C. and stirred for 2 hours. Hydrogen peroxide (24 mL of a 30% aq. solution) is then added, and the reaction mixture is warmed to room temperature and stirred for a further 1 hour. The reaction is cooled back to 0° C. and carefully quenched with a 10% aq. Na₂S2O₃ solution (70 ml). The resulting mixture is extracted with EtOAc (2x), and the combined organic extracts are dried (Na₂SO₄), filtered, and evaporated in vacuo to give a yellow/brown oil. Purification by silica gel column chromatography (25% EtOAc/Hex) affords the title compound as a white solid (3.5 g, 59%).

c) (8R,9S,13S,14S,17S)-3,17-bis(methoxymethoxy)-13-methyl-7,8,9,11,12,13,14,15,16,17-decahydro-6H-cyclopenta[a]phenanthren-6-one—Dess-Martin Periodinane (9.46 g, 22.3 mmol) is added portionwise to a solution of the compound from b) (7.0 g, 18.6 mmol) in 300 mL of CH₂Cl₂. The resulting reaction mixture stirred at room temperature for 3 hours. The mixture is poured into water and the layers are separated. The aqueous layer is extracted with CH₂Cl₂, and the combined organic extracts are washed with brine, dried (Na₂SO₄), filtered, and evaporated in vacuo to give a gooey, brown solid. Purification by silica gel column chromatography (15% EtOAc/Hex) affords the title compound as a pale yellow, viscous oil (6.0 g, 86%).

d) ethyl 2-(((8R,9S,13S,14S,17S)-3,17-bis(methoxymethoxy)-13-methyl-7,8,9,11,12,13,14,15,16,17-decahydro-6H-cyclopenta[a]phenanthren-6-ylidene)acetate —Triethyl phosphonoacetate (4.1 mL, 20.8 mmol) is added to a mixture of sodium hydride (832 mg, 20.8 mmol) in 25 mL of THF at room temperature. After approximately 10 minutes, a solution of the compound from c) (3.9 g, 10.4 mmol) in 10 mL of THF is added dropwise. The resulting reaction mixture is heated to reflux in a sealed tube for 72 hours. The mixture is concentrated in vacuo and purified by silica gel column chromatography (gradient from 5% EtOAc/Hex to 40% EtOAc/Hex) to give the title compound as a clear, viscous oil (3.4 g, 74%).

e) 2-((8R,9S,13S,14S,17S)-3,17-bis(methoxymethoxy)-13-methyl-7,8,9,11,12,13,14,15,16,17-decahydro-6H-cyclopenta[a]phenanthren-6-ylidene)ethanol—A solution of the compound from d) (3.1 g, 6.97 mmol) in 65 mL of THF is treated with lithium aluminum hydride (5.2 mL, 10.46 mmol, 2 M in THF) dropwise at 0° C. The cold bath is removed, and the reaction mixture is stirred at room temperature for 15 minutes. The reaction is cooled back to 0° C. and quenched by the careful addition of 1.3 mL of water, followed by 2.6 mL of 2N NaOH, and then 1.3 mL of water. The mixture is stirred vigorously until a white solid forms. The mixture is filtered, and the filtrate is concentrated in vacuo to give the title compound as a clear oil (2.8 g, 99%).

f) 2-((6S,8R,9S,13S,14S,17S)-3,17-bis(methoxymethoxy)-13-methyl-7,8,9,11,12,13,14,15,16,17-decahydro-6H-cyclopenta[a]phenanthren-6-yl)acetaldehyde—A mixture of the compound from e) (3.09 g, 7.68 mmol) and 10% Pd/C (500 mg) in 100 mL of ethyl acetate is stirred under 40 psi of H₂ (g) for 5 hours at room temperature. The mixture is filtered through Celite, and the Celite is washed well with ethyl acetate. The filtrate is concentrated in vacuo to give a pale yellow oil (3.1 g). The oil is dissolved in 100 mL of dichloromethane, and Dess-Martin Periodinane (3.9 g, 9.22 mmol) is added portionwise. The resulting reaction mixture is stirred at room temperature for 30 minutes. The mixture is poured into water and extracted with CH₂Cl₂. The combined organic extracts are washed with brine, dried (Na₂SO₄), filtered, and evaporated in vacuo to give a brown solid. Purification by silica gel column chromatography (15% EtOAc/Hex) affords the title compound as a clear oil (2.0 g, 65%).

g) 4-((6R,8R,9S,13S,14S,17S)-3,17-bis(methoxymethoxy)-13-methyl-7,8,9,11,12,13,14,15,16,17-decahydro-6H-cyclopenta[a]phenanthren-6-yl)but-2-en-1-ol —Lithium bis(trimethylsilyl)amide (18.4 mL, 18.4 mmol, 1.0 M in THF) is added dropwise to a suspension of (2-hydroxyethyl)triphenylphosphonium bromide (3.37 g, 8.70 mmol) in 60 mL of THF at 0° C. After 1 hour, the golden brown solution is treated with a solution of the compound from f) (1.4 g, 3.48 mmol) in 10 mL of THF dropwise. The resulting reaction mixture is stirred at 0° C. for 40 minutes and then quenched with saturated aqueous NH₄Cl. The resulting mixture is extracted with EtOAc (2x), and the combined organic extracts are dried (Na₂SO₄), filtered, and evaporated to give a brown oil. Purification by silica gel column chromatography (gradient from 20% EtOAc/Hex to 75% EtOAc/Hex) affords the title compound as a yellow, viscous oil (680 mg, 45%).

h) 4-((6R,8R,9S,13S,14S,17S)-3,17-bis(methoxymethoxy)-13-methyl-7,8,9,11,12,13,14,15,16,17-decahydro-6H-cyclopenta[a]phenanthren-6-yl)but-2-enal Dess-Martin Periodinane (437 mg, 1.03 mmol) is added to a solution of the compound from g) (370 mg, 0.86 mmol) in 15 mL of CH₂Cl₂ at room temperature. The resulting reaction mixture is stirred for 10 minutes and then poured into water. The layers are separated and the aqueous layer is extracted with CH₂Cl₂ (2x). The combined organic extracts are washed with brine, dried (Na₂SO₄), filtered, and evaporated in vacuo to give a brown oil. Purification by silica gel column chromatography (gradient from 5% EtOAc/CH2Cl2 to 10% EtOAc/CH₂Cl₂) affords the title compound as a pale yellow, viscous oil (358 mg, 86%).

i) 6-((6R,8R,9S,13S,14S,17S)-3,17-bis(methoxymethoxy)-13-methyl-7,8,9,11,12,13,14,15,16,17-decahydro-6H-cyclopenta[a]phenanthren-6-yl)hexa-2,4-dien-1-ol—Lithium bis(trimethylsilyl)amide (4.3 mL, 4.29 mmol, 1.0 M in THF) is added dropwise to a suspension of (2-hydroxyethyl)triphenylphosphonium bromide (786 mg, 2.03 mmol) in 14 mL of THF at 0° C. After 30 minutes, the golden brown solution is treated with a solution of the compound from h) (345 mg, 0.81 mmol) in 2 mL of THF dropwise. The resulting reaction mixture is stirred at 0° C. for 20 minutes and quenched with saturated aqueous NH₄Cl. The resulting mixture is extracted with EtOAc (2x), and the combined organic extracts are dried (Na₂SO₄), filtered, and evaporated to give a brown oil. Purification by silica gel column chromatography (gradient from 5% EtOAc/CH₂Cl₂ to 40% EtOAc/CH₂Cl₂) affords the title compound as a yellow, viscous oil (140 mg, 38%).

j) (6R,8R,9S,13S,14S,17S)-6-(6-methoxyhexa-2,4-dien-1-yl)-3,17-bis(methoxymethoxy)-13-methyl-7,8,9,11,12,13,14,15,16,17-decahydro-6H-cyclopenta[a]phenanthrene—A solution of the compound in i) (135 mg, 0.3 mmol) is cooled to 0° C., and sodium hydride (120 mg, 3.0 mmol) is added portionwise. After 5-10 minutes, iodomethane (0.19 mL, 3.0 mmol) is added dropwise, and the resulting reaction mixture is warmed to room temperature and stirred for 4 hours. EtOAc is added and the reaction is carefully quenched with water. The layers are separated and the organic layer is dried (Na₂SO₄), filtered, and evaporated to give a brown oily residue. Purification by silica gel column chromatography (gradient from 5% EtOAc/Hex to 20% EtOAc/Hex) affords the title compound as a clear oil (92 mg, 65%).

k) (6R,8R,9S,13S,14S,17S)-6-(6-methoxyhexyl)-3,17-bis(methoxymethoxy)-13-methyl-7,8,9,11,12,13,14,15,16,17-decahydro-6H-cyclopenta[a]phenanthrene—A mixture of the compound in j) (90 mg, 0.19 mmol) and 10% Pd/C (100 mg) in 5-10 mL of ethyl acetate is stirred under a balloon of H₂ (g) for 16 hours at room temperature. The mixture is filtered through Celite, and the Celite is washed well with ethyl acetate. The filtrate is concentrated in vacuo to give the title compound as a clear oil (90 mg, 99%).

l) (6R,8R,9S,13S,14S)-6-(6-methoxyhexyl)-13-methyl-7,8,9,11,12,13,14,15,16,17-decahydro-6H-cyclopenta[a]phenanthrene-3,17-diol (Compound 21)—A solution of the compound from k) (90 mg, 0.19 mmol) in 1.5 mL each of 6 N HCl and THF is stirred for 5 hours at room temperature. The reaction mixture is diluted with water and extracted with EtOAc (2x). The combined organic extracts are dried (Na₂SO₄), filtered, and evaporated in vacuo to give a clear, oily residue. Purification by silica gel column chromatography (gradient from CH2Cl2 to 30% EtOAc/CH2Cl2) afforded Compound 21 as a white solid foam (38 mg, 52%).

### Example 2

### Methods for Preparing Compounds 3 and 4

As outlined in Scheme 2, estradiol derivatives compounds 3 and 4 are synthesized in the following manner. The protected estradiol compound is prepared by reaction of β-estradiol with dihydropyran in THF, using toluenesulfonic acid or camphorsulfonic acid as catalyst. As one of ordinary skill in the art can appreciate, this reaction is an equilibrium reaction and does not go to completion under such conditions. Thus, both the mono-protected estradiols can be found in the reaction mixture. Such crude reaction mixture undergoes a trituration step with acetonitrile causing the desired bis-THP estradiol to crystallize in approximately 70% yield.

As shown in Scheme 2, the key intermediate is obtained via acylation at the benzylic 6-position with the strong base mixture referred to as LiDAKOR: butyl lithium, diisopropylamine, and potassium tert-amylate. Under such conditions at −70° C., one of ordinary skill in the art can appreciate the abstraction of a proton at a benzylic position. The intermediate is then purified by column chromatography to give a syrup in approximately 50% yield, still containing minor impurities and column solvents. Reduction of the aldehyde with an excess of lithium aluminum hydride results in high yields of the racemic hydroxymethyl estradiol compound.

For purposes of preparing compounds 3 and 4, the methoxymethyl compound is prepared by methylation of hydroxymethyl estradiol compound with sodium hydride and methyl iodide. The methoxymethyl compound is purified by column chromatography to give a glassy foam. Deprotecting the protected groups give racemic 6-methoxymethyl estradiol compound. Separation of the enantiomers is performed using chiral preparative HPLC to give the compounds 3 and 4. For compound 4, a chiral purity of >95:5 R:S is realized. For compound 3, a chiral purity of 86:14 S:R is realized. It is well within the level of one of ordinary skill in the art to employ NMR for determination of the absolute stereochemistry of the 6-position, where the 4- and 6-protons are diagnostic.

### Example 3

### Expression Profiling of Compounds in Lung, Pancreas, and Ovarian Tumor Cell Lines

The study includes three human tumor cell lines: A549, Panc-1, and SK-OV-3. The lines are each grown in two flasks cultured to roughly 40% confluence. One of the flasks is treated by addition of compound to the culture media at a various concentrations, i.e. 10 μM, 20 μM 50 μM, or 100 μM. The other, mock treated, flask is treated only with the vehicle used to solubilize and deliver the drug. RNA extracted from the pairs of treated and untreated samples is subjected to microarray analysis on Agilent Whole Human Genome Microarrays (G4112F). Each analysis reports the difference in abundance of messenger RNAs for each of the 41,000 specific mRNA detectors on the array. This direct comparison of the treated versus untreated samples for each cell line provides extremely sensitive detection of changes in mRNA abundance resulting from the drug treatment. As each cell line comparison is self-normalized, the results can be compared across the samples with high confidence.

### Cell Preparation

Three human tumor cell lines, A549, Panc-1, and SK-OV-3, are each grown in two flasks cultured to roughly 40% confluence. One of the flasks is treated by addition of compound to the culture media at concentrations of 10 μM, 50 μM and 100 μM. The other, mock treated, flask is treated only with the vehicle used to solubilize and deliver the drug. All flasks are cultured for a further 24 hours, and then the cells are scraped free and washed in ice-cold PBS, then collected by centrifugation. The harvested cells are immediately frozen, and stored at −80° C. or colder.

### RNA Purification

Total RNA is prepared from the frozen tissue samples using Trizol-based cell lysis followed by 65° C. hot phenol extraction and RNeasy chromatography purification. The purified RNA samples are analyzed spectrophotometrically. The concentration of RNA is determined by measuring the absorbance at 260 nm (A260). Given an absorbance of 1 unit at 260 nm corresponds to 35 gig of RNA per ml when measured at pH 11.

### RNA Quality Assessment—A260/A280 Absorbance Ratios

The ratio of the readings at 260 nm and 280 nm (A260/A280) provides an estimate of the purity of RNA with respect to contaminants that absorb UV, such as protein. RNA has a theoretical A260/A280 ratio (10 mM Tris.Cl, pH 7.5) of approximately 2.1. Extracted RNAs having an A260/A280 ratio of 1.8 or greater provide excellent results in this assay.

### RNA Quality Assessment—Capillary Electrophoresis

The RNA is tested for relative integrity by determining the ratio of intact 28S and 18S ribosomal RNAs, using capillary electrophoresis (Agilent BioAnalyzer). Completely intact RNA has a 28S/18S ratio of 2.2. All RNAs accepted for array analysis have ratios exceeding 1, the minimal 28S/18S ratio for reliably reproducible microarray results as determined by review of internal reproducibility among samples with varying 28S/18S ratios.

### Probe Production and Chip Hybridization

All RNAs are labeled using 1 microgram of RNA as input to an Agilent Low Input Labeling reaction.

Test RNA is labeled with Cy5 (650 nm emitter) and reference RNA is labeled with Cy3 (550 nm emitter) nucleotides. Labeling, hybridizations and subsequent washings are carried out on Agilent H1Av2 human expression chips. The resulting hybridized chips are scanned on an Agilent microarray scanner, and intensity information for each detector spot is extracted from the scanned image using Agilent feature extraction software.

The most telling test of the quality of the hybridization is the level of variance in reported ratios from the large number of duplicates of genes printed on these chips. A set of gene probes is each printed ten times in random positions across the array. The median value of the standard deviation of the log₂ ratio across all the sets is used as an estimator of the overall standard deviation across the entire array.

### Data and Analysis

The key data for all three hybridizations is collected in a FileMaker Pro relational database to allow for easy formulation of searches that can identify genes that exhibit particular transcriptional patterns. The data reported are the red (treated) and green (untreated) background-subtracted signals. This is the least modified form of the data. A background “surface” is estimated across the slide, based on numerous probes that are not complementary to human DNA. These serve as estimators of both non-specific binding of labeled cRNA to array surfaces and non-specific binding of labeled cRNA to the immobilized DNA oligomers. Using this information, local noise around each probe is estimated and this is subtracted from the signal found at the area of oligonucleotide deposition for each particular probe feature on the array (gBGSubSignal, rBGSubSignal). The ratio of signal from the RNA of the treated cell and the RNA of the untreated cell is reported both as a direct ratio and as the log₂ ratio (Ratio, Log 2Ratio). Ratios are determined in an iterative process that normalizes the intensities in each channel, so that a scalar is found that maximizes the similarity of intensities of the large number of genes that have nearly identical transcriptional levels, and thus should have ratios very close to 1.

After the ratios have been calculated for the normalized data, the various control and duplicate samples are analyzed to build a model of how reproducible the results are, and how this reproducibility is varies depending on signal strength and noise. With these parameters, an estimate of the likelihood that each ratio could have arisen if the red and green intensities are randomly drawn from a single process that produced the same distribution of intensities is produced. This probability is reported for each sample and is a measure of the probability that the ratio indicates a difference between the treated and untreated signal strengths (PValLogRatio). This probability can be used to threshold the results into changed and unchanged genes. In the database, a threshold of p≦0.001 is used as the cut point for significant change in mRNA abundance between the treated and untreated sample (Sig0.001). This threshold reduces the number of expected false positives to a reasonable level given the ˜40,000 ratios that are being surveyed in each assay. A field that indicates significant change and the direction of the change relative to the untreated sample reduces the result of the assay to a trinary categorical; 1, up regulated relative to untreated, 0, unchanged relative to untreated and −1, down regulated relative to untreated (Tri). Using this representation, one easily constructs searches that identify genes that have changed in any single or multiple sets of experiments.

The gene expression data found in Table 1 below shows that compound 4 and compound 21 up-regulate the LPL and ApoC2 genes, but not the ApoC3 gene. Gene expression values shown in Table 1 are log₂ values and an average of data obtained from three human tumor cell lines (SKOV-3, A549 and Panc-1). A significant change in gene expression is p<0.001. Gene IDs conform to standards developed at the National Center for Biotechnology Information (NCBI) for the Entrez Gene database.

TABLE 1

| | Cmpd 4- | Cmpd 4- | Cmpd 4- | Cmpd 21- | Cmpd 21- | Cmpd 21- |
|---|---|---|---|---|---|---|
| | SKOV-3 | A549 | Panc-1 | SKOV-3 | A549 | Panc-1 |
| Gene | (10 μM) | (10 μM) | (10 μM) | (50 μM) | (50 μM) | (50 μM) |
| Lipoprotein Lipase | 5.17 | 5.18 | 5.38 | 2.77 | 2.95 | 3.90 |
| mRNA (LPL) | | | | (p = 0.008) | (p = 0.0018) | |
| Apolipoprotein C2 (ApoC2) | 6.55 | 9.00 | 7.27 | 3.89 | 7.21 | 6.91 |
| Apolipoprotein C3 (ApoC3) | 0 | 0 | 0 | 0 | 0 | 0 |

The disclosures of all articles and references, including patents, are incorporated herein by reference. The invention and the manner and process of making and using it are now described in such full, clear, concise and exact terms as to enable any person skilled in the art to which it pertains, to make and use the same. All references cited in this specification are incorporated herein by reference. It is to be understood that the foregoing describes preferred embodiments of the present invention and that modifications may be made therein without departing from the spirit or scope of the present invention.

What is claimed is:

1. A method of preventing or treating Alzheimer's Disease comprising administering to a patient in need thereof a therapeutically effective amount of a 6-substituted estradiol derivative of the formula: 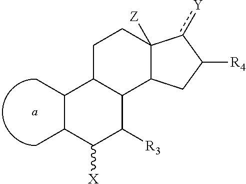
wherein the “a” ring is selected from the group consisting of 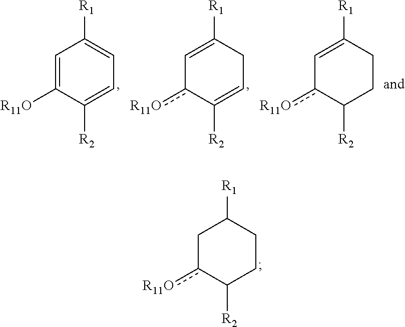
R₁, R₂, R₃ and R₄ are independently selected from the group consisting of H, C₁-C₆ alkyl, halo, a sulfate, a glucuronide, —OH, a bulky group, aryl, cycloalkyl, heteroaryl, heterocycloalkyl, —N(CH₂)ₙ, a phosphate group, and a phosphinate group;
R₁₁ is selected from the group consisting of H, C₁-C₆ alkyl, halogen, a sulfate, a glucoronide, —SO₂NH₂, —COOH, —CN, —CH₂CN—, —NHCN—, —CHO, ═CHOCH₃, —COO salt, —OSO₂alkyl, —NH₂, and —NHCO(CH₂)ₙ;
X is selected from the group consisting of C₁-C₁₂ alkyl, C₂-C₁₂ alkenyl, C₂-C₁₂ alkynyl, halogen, a glucoronide, —NH₂, —SO₂NH₂, —COOH, —CN, —CH₂CN, —NHCN, —CHO, —COOsalt, —OSO₂alkyl, —SH, —SCH₃, —CH[(CH₂)ₙCH₃]COOCH₃, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—NH—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—NH₂, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —N(CH₃)₂, —(CH₂)ₘ(NH)CH₂OH, —NHCOOH, —(CH₂)ₘNHCOOH, —NO₂, —SCN, —SO₂alkyl, —B(OH)₂, —(CH₂)ₘ N(CH₃)—SO₂—NH₃, —(CH₂)ₘ—NH—SO₂—NH₂, —NHC(═S)CH₃, and —NHNH₂;
Y is selected from the group consisting of H, ═O, —OCO(C₁-C₂₀ alkyl) and —OH;
Z is selected from the group consisting of H and methyl;
m is an integer between 0-20;
n is an integer between 0-8;
each [symbol] symbol independently represents either a single or a double bond capable of forming a keto group at position 3 or 17; and
the [symbol] symbol represents any type of bond regardless of the stereochemistry; and the respective enantiomers, other stereochemical isomers, hydrates, solvates, tautomers and pharmaceutically acceptable salts of said compounds.

2. A method according to claim 1 wherein
the “a” ring is 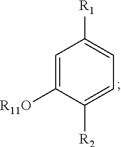
Y is —OH;
Z is methyl;
R₁₁ is H;
R₄ is selected from the group consisting of H, halo and C₁-C₆ alkyl;
R₁ and R₂ are independently selected from the group consisting of H, —OH and halo;
R₃ is selected from the group consisting of H, halo and —OH;
m is an integer from 1-12; and
n is an integer from 0-4.

3. A method according to claim 2 wherein
X is selected from the group consisting of C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃.

4. A method according to claim 1 wherein the compound is selected from the group consisting of (6S,8R,9S,13S,14S)-3-hydroxy-6-(methoxymethyl)-13-methyl-7,8,9,11,12,13,15,16-octahydro-6H-cyclopenta[a]phenanthren-17(14H)-one; (6R,8R,9S,13S,14S)-3-hydroxy-6-(methoxymethyl)-13-methyl-7,8,9,11,12,13,15,16-octahydro-6H-cyclopenta[a]phenanthren-17(14H)-one; (6S,8R,9S,13S,14S)-6-(methoxymethyl)-13-methyl-7,8,9,11,12,13,14,15,16,17-decahydro-6H-cyclopenta[a]phenanthrene-3,17-diol; (6R,8R,9S,13S,14S)-6-(methoxymethyl)-13-methyl-7,8,9,11,12,13,14,15,16,17-decahydro-6H-cyclopenta[a]phenanthrene-3,17-diol; (6S,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one; (6R,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one; (6S,8R,9S,13S,14S)-6-(hydroxymethyl)-13-methyl-7,8,9,11,12,13,14,15,16,17-decahydro-6H-cyclopenta[a]phenanthrene-3,17-diol; (6R,8R,9S,13S,14S)-6-(hydroxymethyl)-13-methyl-7,8,9,11,12,13,14,15,16,17-decahydro-6H-cyclopenta[a]phenanthrene-3,17-diol; (6R,8R,9S,10R,13S,14S)-6-(methoxymethyl)-10,13-dimethylhexadecahydro-1H-cyclopenta[a]phenanthrene-3,17-diol; (6R,8R,9S,13S,14S)-6-((aminooxy)methyl)-13-methyl-7,8,9,11,12,13,14,15,16,17-decahydro-6H-cyclopenta[a]phenanthrene-3,17-diol; (6S,8R,9S,13S,14S)-6-((aminooxy)methyl)-13-methyl-7,8,9,11,12,13,14,15,16,17-decahydro-6H-cyclopenta[a]phenanthrene-3,17-diol; (6R,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one; (6S,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one; (6R,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol; (6S,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol; 1-((((6R,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one; 1-((((6S,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one; (6R,8R,9S,13S,14S)-6-methoxy-13-methyl-7,8,9,11,12,13,14,15,16,17-decahydro-6H-cyclopenta[a]phenanthrene-3,17-diol; (6S,8R,9S,13S,14S)-6-(2-methoxyethyl)-13-methyl-7,8,9,11,12,13,14,15,16,17-decahydro-6H-cyclopenta[a]phenanthrene-3,17-diol; (6R,8R,9S,13S,14S)-6-(4-methoxybutyl)-13-methyl-7,8,9,11,12,13,14,15,16,17-decahydro-6H-cyclopenta[a]phenanthrene-3,17-diol; (6R,8R,9S,13S,14S)-6-(6-methoxyhexyl)-13-methyl-7,8,9,11,12,13,14,15,16,17-decahydro-6H-cyclopenta[a]phenanthrene-3,17-diol; (6R,8R,9S,13S,14S)-6-(6-methoxyoctyl)-13-methyl-7,8,9,11,12,13,14,15,16,17-decahydro-6H-cyclopenta[a]phenanthrene-3,17-diol; (6R,8R,9S,13S,14S)-3-hydroxy-6-(methoxymethyl)-13-methyl-7,8,9,11,12,13,14,15,16,17-decahydro-6H-cyclopenta[a]phenanthren-17-yl stearate; (6R,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione; (6S,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione; (6R,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol; (6S,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol; (6S,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate; (6R,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate; (6R,8R,9S,13S,14S)-13-methyl-6-(4-propoxybutyl)-7,8,9,11,12,13,14,15,16,17-decahydro-6H-cyclopenta[a]phenanthrene-3,17-diol; (6R,8R,9S,13S,14S)-13-methyl-6-(5-ethoxypentyl)-7,8,9,11,12,13,14,15,16,17-decahydro-6H-cyclopenta[a]phenanthrene-3,17-diol; (6R,8R,9S,10R,13S,14S)-6-(methoxymethyl)-10,13-dimethylhexadecahydro-1H-cyclopenta[a]phenanthrene-3,17-diol; and (6R,8S,9S,14S,17S)-6-(methoxymethyl)-7,8,9,11,12,13,14,15,16,17-decahydro-6H-cyclopenta[a]phenanthrene-3,17-diol.

5. A method according to claim 4 wherein the compound is selected from the group consisting of (6R,8R,9S,13S,14S)-6-(methoxymethyl)-13-methyl-7,8,9,11,12,13,14,15,16,17-decahydro-6H-cyclopenta[a]phenanthrene-3,17-diol and (6R,8R,9S,13S,14S)-6-(6-methoxyhexyl)-13-methyl-7,8,9,11,12,13,14,15,16,17-decahydro-6H-cyclopenta[a]phenanthrene-3,17-diol.

6. A method of up-regulation of LPL and/or ApoC2 functional activity in a mammal, said method comprising administering to said mammal an effective amount of a compound of formula: 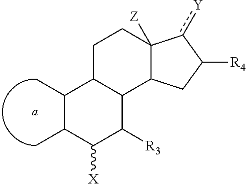
wherein the “a” ring is selected from the group consisting of 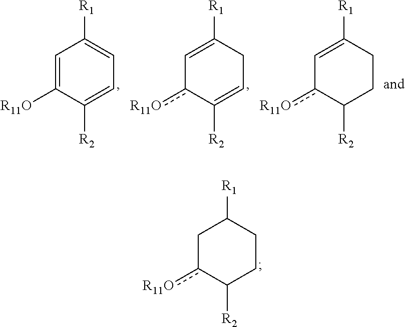
R₁, R₂, R₃ and R₄ are independently selected from the group consisting of H, C₁-C₆ alkyl, halo, a sulfate, a glucuronide, —OH, a bulky group, aryl, cycloalkyl, heteroaryl, heterocycloalkyl, —N(CH₂)ₙ, a phosphate group, and a phosphinate group;
R₁₁ is selected from the group consisting of H, C₁-C₆ alkyl, halogen, a sulfate, a glucoronide, —SO₂NH₂, —COOH, —CN, —CH₂CN—, —NHCN—, —CHO, ═CHOCH₃, —COO salt, —OSO₂alkyl, —NH₂, and —NHCO(CH₂)ₙ;
X is selected from the group consisting of C₁-C₁₂ alkyl, C₂-C₁₂ alkenyl, C₂-C₁₂ alkynyl, halogen, a glucoronide, —NH₂, —SO₂NH₂, —COOH, —CN, —CH₂CN, —NHCN, —CHO, —COOsalt, —OSO₂alkyl, —SH, —SCH₃, —CH[(CH₂)ₙCH₃]COOCH₃, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—NH—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—NH₂, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —N(CH₃)₂, —(CH₂)ₘ(NH)CH₂OH, —NHCOOH, —(CH₂)ₘNHCOOH, —NO₂, —SCN, —SO₂alkyl, —B(OH)₂, —(CH₂)ₘ N(CH₃)—SO₂—NH₃, —(CH₂)ₘ—NH—SO₂—NH₂, —NHC(═S)CH₃, and —NHNH₂;
Y is selected from the group consisting of H, ═O, —OCO(C₁-C₂₀ alkyl) and —OH;
Z is selected from the group consisting of H and methyl;
m is an integer between 0-20;
n is an integer between 0-8;
each [symbol] symbol independently represents either a single or a double bond capable of forming a keto group at position 3 or 17; and
the [symbol] symbol represents any type of bond regardless of the stereochemistry; and the respective enantiomers, other stereochemical isomers, hydrates, solvates, tautomers and pharmaceutically acceptable salts of said compounds.

7. A method according to claim 6 wherein
the “a” ring is 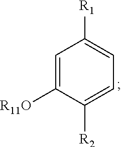
Y is —OH;
Z is methyl;
R₁₁ is H;
R₄ is selected from the group consisting of H, halo and C₁-C₆ alkyl;
R₁ and R₂ are independently selected from the group consisting of H, —OH and halo;
R₃ is selected from the group consisting of H, halo and —OH;
m is an integer from 1-12; and
n is an integer from 0-4.

8. A method according to claim 7 wherein
X is selected from the group consisting of C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃.

9. A method according to claim 6 wherein the compound is selected from the group consisting of (6S,8R,9S,13S,14S)-3-hydroxy-6-(methoxymethyl)-13-methyl-7,8,9,11,12,13,15,16-octahydro-6H-cyclopenta[a]phenanthren-17(14H)-one; (6R,8R,9S,13S,14S)-3-hydroxy-6-(methoxymethyl)-13-methyl-7,8,9,11,12,13,15,16-octahydro-6H-cyclopenta[a]phenanthren-17(14H)-one; (6S,8R,9S,13S,14S)-6-(methoxymethyl)-13-methyl-7,8,9,11,12,13,14,15,16,17-decahydro-6H-cyclopenta[a]phenanthrene-3,17-diol; (6R,8R,9S,13S,14S)-6-(methoxymethyl)-13-methyl-7,8,9,11,12,13,14,15,16,17-decahydro-6H-cyclopenta[a]phenanthrene-3,17-diol; (6S,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one; (6R,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one; (6S,8R,9S,13S,14S)-6-(hydroxymethyl)-13-methyl-7,8,9,11,12,13,14,15,16,17-decahydro-6H-cyclopenta[a]phenanthrene-3,17-diol; (6R,8R,9S,13S,14S)-6-(hydroxymethyl)-13-methyl-7,8,9,11,12,13,14,15,16,17-decahydro-6H-cyclopenta[a]phenanthrene-3,17-diol; (6R,8R,9S,10R,13S,14S)-6-(methoxymethyl)-10,13-dimethylhexadecahydro-1H-cyclopenta[a]phenanthrene-3,17-diol; (6R,8R,9S,13S,14S)-6-((aminooxy)methyl)-13-methyl-7,8,9,11,12,13,14,15,16,17-decahydro-6H-cyclopenta[a]phenanthrene-3,17-diol; (6S,8R,9S,13S,14S)-6-((aminooxy)methyl)-13-methyl-7,8,9,11,12,13,14,15,16,17-decahydro-6H-cyclopenta[a]phenanthrene-3,17-diol; (6R,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one; (6S,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one; (6R,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol; (6S,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol; 1-((((6R,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one; 1-((((6S,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one; (6R,8R,9S,13S,14S)-6-methoxy-13-methyl-7,8,9,11,12,13,14,15,16,17-decahydro-6H-cyclopenta[a]phenanthrene-3,17-diol; (6S,8R,9S,13S,14S)-6-(2-methoxyethyl)-13-methyl-7,8,9,11,12,13,14,15,16,17-decahydro-6H-cyclopenta[a]phenanthrene-3,17-diol; (6R,8R,9S,13S,14S)-6-(4-methoxybutyl)-13-methyl-7,8,9,11,12,13,14,15,16,17-decahydro-6H-cyclopenta[a]phenanthrene-3,17-diol; (6R,8R,9S,13S,14S)-6-(6-methoxyhexyl)-13-methyl-7,8,9,11,12,13,14,15,16,17-decahydro-6H-cyclopenta[a]phenanthrene-3,17-diol; (6R,8R,9S,13S,14S)-6-(6-methoxyoctyl)-13-methyl-7,8,9,11,12,13,14,15,16,17-decahydro-6H-cyclopenta[a]phenanthrene-3,17-diol; (6R,8R,9S,13S,14S)-3-hydroxy-6-(methoxymethyl)-13-methyl-7,8,9,11,12,13,14,15,16,17-decahydro-6H-cyclopenta[a]phenanthren-17-yl stearate; (6R,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione; (6S,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione; (6R,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol; (6S,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol; (6S,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate; (6R,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate; (6R,8R,9S,13S,14S)-13-methyl-6-(4-propoxybutyl)-7,8,9,11,12,13,14,15,16,17-decahydro-6H-cyclopenta[a]phenanthrene-3,17-diol; (6R,8R,9S,13S,14S)-13-methyl-6-(5-ethoxypentyl)-7,8,9,11,12,13,14,15,16,17-decahydro-6H-cyclopenta[a]phenanthrene-3,17-diol; (6R,8R,9S,10R,13S,14S)-6-(methoxymethyl)-10,13-dimethylhexadecahydro-1H-cyclopenta[a]phenanthrene-3,17-diol; and (6R,8S,9S,14S,17S)-6-(methoxymethyl)-7,8,9,11,12,13,14,15,16,17-decahydro-6H-cyclopenta[a]phenanthrene-3,17-diol.

10. A method according to claim 9 wherein the compound is selected from the group consisting of (6R,8R,9S,13S,14S)-6-(methoxymethyl)-13-methyl-7,8,9,11,12,13,14,15,16,17-decahydro-6H-cyclopenta[a]phenanthrene-3,17-diol and (6R,8R,9S,13S,14S)-6-(6-methoxyhexyl)-13-methyl-7,8,9,11,12,13,14,15,16,17-decahydro-6H-cyclopenta[a]phenanthrene-3,17-diol.
